SEQUENCE LISTING (2 sequences)

SEQ ID NO: 1 - DNA, 616 bp, Homo sapiens
  CDS (26)...(616)
aggagagcataccacctggagcaagatggatgtggggagcaaagaggtcctgatggagagcccgccggactactccgcagctccccggggccgatttggcattccctgctgcccagtgcacctgaaacgccttcttatcgtggtggtggtggtggtcctcatcgtcgtggtgattgtgggagccctgctcatgggtctccacatgagccagaaacacacggagatggttctggagatgagcattggggcgccggaagcccagcaacgcctggccctgagtgagcacctggttaccactgccaccttctccatcggctccactggcctcgtggtgtatgactaccagcagctgctgatcgcctacaagccagcccctggcacctgctgctacatcatgaagatagctccagagagcatccccagtcttgaggctctcaatagaaaagtccacaacttccagatggaatgctctctgcaggccaagcccgcagtgcctacgtctaagctgggccaggcagaggggcgagatgcaggctcagcaccctccggaggggacccggccttcctgggcatggccgtgaacaccctgtgtggcgaggtgccgctctactacatc

SEQ ID NO: 2 - protein, 197 aa, Homo sapiens
MDVGSKEVLMESPPDYSAAPRGRFGIPCCPVHLKRLLIVVVVVVLIVVVIVGALLMGLHMSQKHTEMVLEMSIGAPEAQQRLALSEHLVTTATFSIGSTGLVVYDYQQLLIAYKPAPGTCCYIMKIAPESIPSLEALNRKVHNFQMECSLQAKPAVPTSKLGQAEGRDAGSAPSGGDPAFLGMAVNTLCGEVPLYYI

(19) United States
(12) Patent Application Publication
(10) Pub. No.: US 2002/0197646 A1
(43) Pub. Date: Dec. 26, 2002

(54) Single nucleotide polymorphisms associated with interstitial lung disease

(76) Inventors: Lawrence M. Nogee, Towson, MD (US); F. Sessions Cole, St. Louis, MO (US); Jeffrey A. Whitsett, Cincinnati, OH (US); Aaron Hamvas, St. Louis, MO (US)

Correspondence Address: BANNER & WITCOFF, 1001 G STREET N W, SUITE 1100, WASHINGTON, DC, 20001, US

(21) Appl. No.: 10/074,247

(22) Filed: Feb. 14, 2002

Related U.S. Application Data
(60) Provisional application No. 60/268,650, filed on Feb. 14, 2001.
(60) Provisional application No. 60/268,991, filed on Feb. 15, 2001.

(51) Int. Cl. G01N 33/53; C07K 16/00

(52) U.S. Cl. 435/7.1; 530/388.1

(57) ABSTRACT

Single nucleotide polymorphisms (SNPs) in the gene encoding surfactant protein C can be used to diagnose interstitial lung disease and to determine whether an individual is predisposed to developing interstitial lung disease. Single-stranded polynucleotides comprising a contiguous series of nucleotides from a mutant allele of a surfactant protein C gene, as well as antibodies which specifically bind to altered forms of surfactant protein C but not to wild-type surfactant protein C, can be used in various methods to detect the presence of disease-associated SNPs.

This application claims priority to and incorporates by reference co-pending provisional applications Serial No. 60/268,650 filed Feb. 14, 2001 and Serial No. 60/268,991 filed Feb. 15, 2001.

This invention resulted from research funded in whole or in part by National Institutes of Health Grant Nos. HL54703 and HL54187. The Federal Government has certain rights in this invention.

### FIELD OF THE INVENTION

The invention relates to single nucleotide polymorphisms that are associated with interstitial lung diseases and/or pulmonary fibrosis or with predisposition to these diseases.

### BACKGROUND OF THE INVENTION

Interstitial lung diseases (ILD) are a heterogeneous group of disorders that include interstitial pneumonias with variable degrees and types of pulmonary inflammation and that are poorly understood at a molecular level (1, 2). The cause of ILD is often unknown. Moreover, the histological diagnoses used in adults may represent different disease processes in children (3-5). For example, desquamative interstitial pneumonitis (DIP) is often more severe and refractory to treatment in young infants than in adults (6, 7). DIP in infants may represent a more recently described form of ILD termed chronic pneumonitis of infancy (CPI) (8, 9). The lung pathology in CPI is characterized by interstitial thickening with mesenchymal cells, as opposed to an inflammatory infiltrate, and an alveolar infiltrate of cellular accumulation with variable amounts of proteinaceous material. A possible genetic basis for DIP and CPI is suggested by familial cases (6, 8, 10).

Pulmonary surfactant is the mixture of lipids and proteins needed to reduce surface tension and prevent end expiratory atelectasis. Deficiency of pulmonary surfactant is the principal cause of respiratory distress syndrome (RDS) in premature infants (25). Surfactant protein B (SP-B) and surfactant protein C (SP-C) are hydrophobic proteins that enhance the surface tension lowering properties of surfactant lipids, and both are present in lung derived surfactant preparations used to treat infants with RDS (26). The inability to produce SP-B causes lethal neonatal lung disease in both genetically engineered mice and in human infants homozygous for mutations in the SP-B gene (12, 13, 27).

Lung disease also may result from deficiency of mature SP-C. Genetically engineered mice incapable of producing SP-C survive into adulthood, but have abnormal surfactant that is unstable at low lung volumes. Deficiency of SP-C could thus predispose individuals to recurrent atelectasis, lung injury, and inflammation. The lack of SP-C may have secondary effects on metabolism and function of other surfactant components, or SP-C may have an as yet unknown but essential function.

Mature SP-C is derived by proteolytic processing of a 197 amino acid (or, if alternative splicing occurs, a 191 amino acid) proprotein (14-16). ProSP-C is an integral membrane protein, with the hydrophobic core of mature SP-C anchoring it in the membrane (28). SP-C can form oligomers and interacts with surfactant phospholipids and SP-B (31). Interactions between normal and abnormal proSP-C could hinder the transit of normal proSP-C through the processing pathway or enhance its degradation. Competitive inhibition by the abnormal proprotein could also interfere with processing of normal proSP-C. Deletions in the carboxyterminal domain have been shown to disrupt intracellular trafficking of proSP-C (29, 30).

Several mechanisms may relate abnormalities in SP-C metabolism to the development of lung disease. Accumulation or misrouting of improperly folded proteins has been increasingly recognized as causing disease, including α1-antitrypsin deficiency and cystic fibrosis (34). An abnormal SP-C proprotein is unlikely to have folded properly. Because SP-C is extremely hydrophobic, improperly folded proSP-C may result in aggresome formation, secondary cellular injury, and subsequent inflammation (32). Because the expression and processing of SP-C are developmentally regulated (33), the postnatal onset of lung disease could be related to increased expression or accumulation of abnormal proSP-C. Agents that enhance intracellular processing and transport of misfolded proteins may have a role in therapy of ILD (35, 36). In addition, patients with defective SP-C proteins may be refractory to certain therapeutic treatments. Thus, there is a need in the art for reagents and methods for diagnosing and screening for predisposition to interstitial lung diseases associated with abnormalities in SP-C or its proprotein.

### BRIEF SUMMARY OF THE INVENTION

It is an object of the invention to provide reagents and methods for diagnosing and screening for predisposition to interstitial lung diseases associated with abnormalities in surfactant protein C. These and other objects of the invention are provided by one or more of the embodiments described below.

One embodiment of the invention is a purified preparation of antibodies which specifically bind to a mutant human surfactant protein C comprising an amino acid alteration due to the presence of a single nucleotide polymorphism (SNP) in a gene encoding the mutant surfactant protein C. The SNP is associated with interstitial lung disease. The antibodies do not bind to a wild-type human surfactant protein C.

Another embodiment of the invention is a single-stranded polynucleotide comprising 12 contiguous nucleotides of a mutant allele of a human surfactant protein C gene. The 12 contiguous nucleotides comprise a SNP which is associated with interstitial lung disease.

Yet another embodiment of the invention is a kit comprising a reagent for detecting a SNP in a mutant allele of a human surfactant protein C gene and instructions for a method of detecting the SNP. The SNP is associated with interstitial lung disease.

Still another embodiment of the invention is a method of identifying an individual as predisposed to developing interstitial lung disease associated with a defect in surfactant protein C. A biological sample obtained from the individual is assayed to determine if an allele of a surfactant protein C gene comprises a SNP associated with interstitial lung disease. The individual is identified as predisposed to developing the interstitial lung disease if the allele comprises the SNP.

Even another embodiment of the invention is a method of diagnosing interstitial lung disease associated with a defect in surfactant protein C. A biological sample obtained from an individual is assayed to determine if an allele of a surfactant protein C gene comprises a SNP associated with interstitial lung disease. The individual is identified as having the interstitial lung disease if the allele comprises the SNP.

A further embodiment of the invention is a method of determining whether an individual having interstitial lung disease is likely to respond to a therapeutic intervention. A biological sample obtained from the individual is assayed to determine whether both alleles of the individual's surfactant protein C gene comprise a SNP associated with interstitial lung disease. The individual is identified as likely to respond to the therapeutic intervention if neither allele comprises the SNP.

### BRIEF DESCRIPTION OF THE FIGURES

FIGS. 1A-1D. Immunohistochemical staining for proSP-C. Without antigen retrieval, staining for proSP-C was undetectable in case patient A (FIG. 1A) and weak to absent in the patient's mother (FIG. 1B). Following antigen retrieval, robust staining for proSP-C was observed in both the case patient (FIG. 1C) and her mother (FIG. 1D), with proSP-C staining confined to the alveolar epithelium. Robust staining for proSP-C was detected in controls without the need for antigen retrieval. Original magnification=230X for all panels.

FIGS. 2A-2C. Immunoblotting for surfactant proteins in lung tissue. FIG. 2A, Mature SP-B was detected in lung tissue from all patients, except in an infant with a known hereditary SP-B deficiency. FIG. 2B, ProSP-C in tissue from the case patient was reduced in amount, and its migration corresponded to a lower molecular weight than proSP-C in tissues from control patients. FIG. 2C, Mature SP-C was detected in control lung tissues, but not in tissue from the case patient. Aberrantly processed proSP-C peptides, characteristically found in lung tissue of infants with hereditary SP-B deficiency mutations (arrows on left) (13, 22), were not observed in lung tissue from the patient with an SP-C gene mutation or in control patients. Control sample 1 was from a patient with pulmonary hypertension. Control samples 2 and 3 were from normal lung tissue. Results shown are representative of at least three separate experiments.

FIGS. 3A-3B. DNA analyses. FIG. 3A, the arrow points to a heterozygous substitution immediately after the last base of codon 145 (the first base in intron 4) in the case patient's SP-C gene DNA sequence, which eliminated the invariant G in the normal splice donor consensus sequence. FIG. 3B, restriction analysis. The c.460+1 G>A mutation eliminates a restriction site for the enzyme BstN1 (top). After PCR amplification of the region containing the mutation and digestion of the PCR products with BstN1, the presence of a 126 bp band in the lanes from the mother and child indicate that both carried the mutation on one allele. Arrows on top figure indicate the locations of the inner primers (g.1564-g.1582, forward and g.1778-g.1757, reverse) used in the nested PCR reactions.

FIG. 4. RT-PCR for SP-C cDNA. Two different sized SP-C cDNAs were amplified from the case patient, the smaller one corresponding to the size of the deletion of exon 4 sequence.

### DETAILED DESCRIPTION OF THE INVENTION

We have identified single nucleotide polymorphisms (SNPs) in the human surfactant protein C (SP-C) gene. These SNPs and the amino acids corresponding to the SNPs are listed in Tables 1 and 2. Genomic sequences of wild-type human surfactant protein C (SP-C) genes are disclosed in Glasser et al., J. Biol. Chem. 263, 10326-31, 1988; a wild-type cDNA sequence is disclosed in Warr et al., Proc. Natl. Acad. Sci. U.S.A. 84, 715-19, 1987 and in SEQ ID NO:1. Wild-type human ProSP-C is shown in SEQ ID NO:2. As used herein, “SP-C protein” includes both the pro-protein and the processed form of the protein.

The SNP at position 1728, referred to in the Examples below as c.460+1G>A or c.460+1G>T, has been demonstrated to be associated with expression of an abnormal SP-C proprotein and undetectable amount of mature SP-C in a patient with familial ILD. This mutation results in the deletion of 37 amino acids in the carboxyterminal domain of proSP-C.

Detection of these SNPs can be used to identify individuals who have a predisposition for developing an interstitial lung disease, such as chronic pneumonitis of infancy, respiratory distress syndrome, recurrent atelectasis, interstitial lung injury, arcoidosis, pulmonary alveolar proteinosis, disorders associated with lung inflammation (e.g., progressing to idiopathic pulmonary fibrosis, diffuse interstitial penumonitis, hypersensitivity pneumonitis, hypersensitivity fibrosis, or pulmonary fibrosis of collagen and vascular diseases), idiopathic pulmonary fibrosis (e.g., usual interstitial pneumonitis, desquamative interstitial pneumonitis, acute interstitial pneumonitis, and non-specific interstitial pneumonitis), secondary pulmonary fibrosis, hypersensitivity pneumonitis, bronchiolitis obliterans, cryptogenic organizing pneumonia, pulmonary Langerhans cell granulomatosis, pulmonary sarcoidosis, and lymphangitic carcinomatosis. For this purpose, the invention provides single-stranded polynucleotides, which can be used as probes and primers for use in detecting the SNPs themselves. The invention also provides antibodies for use in detecting the amino acid alterations in the SP-C protein corresponding to the SNP.

SNPs in the human SP-C gene can arise either from germline or somatic mutations. Screening methods of the invention are particularly useful in combination with a subject's family history. To alleviate a parent's concern and to take any preventative measures which might prevent or delay onset of interstitial lung disease, one or more of the screening methods disclosed herein can be used to determine whether a child is a carrier of one or more disease-associated SNPs. The screening methods of the invention also can be used in combination with existing methods for diagnosing lung injury (e.g., radiological or biochemical) to maximize confidence in the ultimate diagnosis.

The natural history and response to different therapeutic agents, such as anti-inflammatory agents (e.g., glucocorticoids) and chloroquine, are variable in childhood ILD, and may depend in part upon etiology. Individuals with genetic causes may be less likely to respond to these therapies. The identification of SP-C gene mutations associated with interstitial lung disease can provide a classification of this set of diseases, as well as for diagnosis by molecular methods without the need for invasive procedures, such as lung biopsy. Thus, the invention also provides methods of identifying individuals who may benefit from a particular therapeutic intervention, sparing those for whom the intervention may not be effective from undergoing unnecessary treatments.

TABLE 1

| | | | | | | Nucleotide |
|---|---|---|---|---|---|---|
| Genomic | Wild- | | Gene | Amino Acid | | of SEQ |
| Location | Type | Variant | Location | Alteration | Codon | ID NO:1 |
| 49+ | C | G | Exon 1 | Val > Val | 8 | 49 |
| 99 | C | T | Intron 1 | | | |
| 102 | G | A | Intron 1 | | | |
| 106 | A | G | Intron 1 | | | |
| 493 | G | A | Intron 1 | | | |
| 574 | A | G | Intron 1 | | | |
| 745 | T | C | Intron 1 | | | |
| 812+ | C | T | Exon 2 | Pro > Leu | 30 | 114 |
| 938 | G | A | Intron 2 | | | |
| 1287+ | T | C | Exon 3 | Ile > Thr | 73 | 243 |
| 1368+ | G | T | Exon 3 | Gly > Val | 100 | 324 |
| 1376+ | G | C | Exon 3 | Val > Leu | 103 | 332 |
| 1379+ | T | C | Exon 3 | Tyr > His | 104 | 335 |
| 1636+ | C | T | Exon 4 | Pro > Leu | 115 | 369 |
| 1669+ | T | G | Exon 4 | Ile > Arg | 126 | 402 |
| 1705* | C | A | Exon 4 | Thr > Asn | 138 | |
| 1712+ | A | Deletion | Exon 4 | Frameshift | 140 | 443-445 |
| 1728+ | G | A | Intron 4 | Skips exon 4 | | 460 + 1 |
| 1728+ | G | T | Intron 4 | Skips exon 4 | | 460 + 1 |
| 1798 | G | A | Intron 4 | | | |
| 1875 | C | T | Intron 4 | | | |
| 2056 | C | G | Intron 4 | | | |
| 2185* | G | A | Exon 5 | Ser > Thr | 186 | |
| 2188+ | C | A | Exon 5 | Thr > Asn | 187 | 585 |
| 2191+ | T | G | Exon 5 | Leu > Arg | 188 | 588 |

*Previously reported
+Only in patients with lung disease

TABLE 2

| | | | | | | Nucleotide |
|---|---|---|---|---|---|---|
| Genomic | Wild- | | Gene | Amino Acid | | of SEQ ID |
| Location | Type | Variant | Location | Alteration | Codon | NO:1 |
| 917+ | C | A | Exon 2 | Thr > Lys | 65 | 219 |
| 973 | A | C | Intron 2 | | | |
| 1290+ | G | T | Exon 3 | Gly > Val | 74 | 246 |
| 1503 | C | T | Intron 3 | | | |
| 1626+ | G | A | Exon 4 | Ala > Thr | 112 | 359 |
| 1957 | C | T | Intron 4 | | | |
| 2126+ | G | insertion | Exon 5 | Frameshift | 166 | 521-523 |
| | | of G | | | | |

+Only in patients with lung disease

Reagents Useful in Methods of Detecting Mutant SP-C Alleles

The invention provides reagents, including single-stranded polynucleotides and antibodies, which can be used in screening methods of the invention. Any of these reagents can be provided in kits, together with instructions for their use. Kits may also include other components, such as appropriate buffers, detecting reagents, reaction vessels, single or divided containers, and solid supports, such as a gene chip, glass or plastic slide, tissue culture plate, microtiter well, tube, silicon chip, or particle, such as a bead (e.g., a latex, polystyrene, or glass bead).

Single-Stranded Polynucleotides

A genomic coding sequence for human wild-type SP-C is shown in SEQ ID NO:1. SNPs which have been identified in this sequence are shown in Table 1. A “mutant allele” of a human SP-C gene comprises at least one SNP with respect to SEQ ID NO:1, which is associated with interstitial lung disease. Association of a SNP with interstitial lung disease can be determined, for example, by statistical correlation of a disease phenotype with a particular SNP. Such methods are well known in the art. See, e.g., Curtis et al., Ann Hum Genet 2001 Jan;65(Pt 1):95-107 Johansson et al., Genes Immun 2000 Aug; 1 (6):380-5; Johnson et al., Genes Immun 2001 Aug;2(5):273-5.

Single-stranded polynucleotides of the invention comprise at least 12 contiguous nucleotides of a mutant SP-C allele, provided that the SNP is located within the 12 contiguous nucleotides. Single-stranded polynucleotides can contain any length from 12 contiguous nucleotides to a full-length sequence (e.g., 12, 13, 14, 15, 20, 25, 50, 75, 100, 250, or 500 or more contiguous nucleotides). The SNP can be any of the 12 contiguous nucleotides, including the nucleotide at either the 3′ or the 5′ end of the molecule.

Single-stranded polynucleotides of the invention can be used as probes or as primers. Pairs of primers can be used to amplify SNP-containing portions of a mutant SP-C allele. Probes can comprise a detectable label, such as a fluorescent, chemiluminescent, or radioactive label.

Antibodies

The invention provides antibodies which specifically bind to mutant human SP-C proteins. A “mutant human SP-C protein” comprises an amino acid alteration due to the presence of a SNP in the human SP-C gene as shown in SEQ ID NO:1, which is associated with interstitial lung disease. Mutant human SP-C proteins include those with the amino acid alterations disclosed in Tables 1 and 2.

“Antibody” as used herein includes intact immunoglobulin molecules, as well as fragments, such as Fab, F(ab′)2, and Fv fragments, which are capable of binding an epitope of such a mutant SP-C protein. Typically, at least 6, 8, 10, or 12 contiguous amino acids are required to form an epitope. However, epitopes which involve non-contiguous amino acids may require more, e.g., at least 15, 25, or 50 amino acids. Purified antibody preparations of the invention are those in which a majority of the antibodies present in the preparation specifically bind to a mutant human SP-C protein.

An antibody which specifically binds to an epitope of a mutant human SP-C protein can be used in immunochemical assays, such as Western blots, ELISAs, radioimmunoassays, immunohistochemical assays, immunoprecipitations, or other immunochemical assays known in the art. Various immunoassays can be used to identify antibodies having the desired specificity. Numerous protocols for competitive binding or immunoradiometric assays are well known in the art. Such immunoassays typically involve the measurement of complex formation between an antigen and an antibody that specifically binds to the antigen.

Typically, an antibody which specifically binds to a mutant human SP-C protein provides a detection signal at least 5-, 10-, or 20-fold higher than a detection signal provided with other proteins when used in an immunochemical assay. Preferably, antibodies which specifically bind to a mutant human SP-C protein do not detect wild-type SP-C protein or other proteins in immunochemical assays and can immunoprecipitate the mutant human SP-C protein from solution but not wild-type SP-C protein.

Screening Methods for Detecting SNPs

Frequently, a polymorphism itself is not causative of disease predisposition, but is linked to sequences that result in disease predisposition. In other cases, the SNP itself may affect gene expression. The use of SNP markers for genotyping is well documented. See, e.g., Mansfield et al., Genomics 24, 225-33, 1994; Ziegle et al., Genomics 14, 1026-31, 1992. SNPs of the present invention are associated with interstitial lung disease or injury. The invention provides methods of screening for interstitial lung diseases or injury or for a predisposition for developing an interstitial lung disease or injury by assaying a biological sample for the presence or absence of one or more SNPs in the human SP-C gene, such as the SNPs listed in Table 1.

Biological Samples

For examination of nucleic acids, the biological sample can be any conveniently obtained sample, such as a cord blood sample, biopsy material, parental blood, etc. For prenatal diagnosis, fetal nucleic acid samples can be obtained from maternal blood as described in WO 91/07660. Alternatively, amniocytes or chorionic villi can be obtained for use in prenatal testing. Biological samples also include biological fluids such as bronchoalveolar lavage fluid, tracheal lavage fluid, cerebrospinal fluid, tears, saliva, lymph, dialysis fluid, organ or tissue-culture derived fluids, and fluids extracted from biological tissues. Other biological samples include cells dissociated from a tissue sample or histological sections. of a tissue Alternatively, a lysate of the cells may be prepared.

For examination of SP-C protein itself, the protein can be purified from type II alveolar epithelial cells using standard protein purification methods. Alternatively, a lysate of type II alveolar epithelial cells can be prepared.

Amplification of Nucleic Acids to be Tested

Amplification of nucleic acids can be carried out using conventional methods. See, e.g., Maniatis et al., MOLECULAR CLONING: A LABORATORY MANUAL, pages 187-210 (Cold Spring Harbor Laboratory, 1982). For example, mRNA from alveolar type II cells can be converted to cDNA and then enzymatically amplified to produce microgram quantities of cDNA encoding SP-C. Preferably, amplification is carried out using the polymerase chain reaction (“PCR”) method (see U.S. Pat. Nos. 4,698,195, 4,800,159, 4,683,195, and 4,683,202). Sequences complementary to primer pairs can be separated by as many nucleotides as the amplification technique will allow. However, one of skill in the art will understand that there are practical limitations of subsequent assaying procedures, which may dictate the number of nucleotides between the sequences complementary to the primer pairs. In one embodiment, the primers are equidistant from the nucleotide(s) targeted for amplification.

Alternative amplification methods include the ligase chain reaction (“LCR”) (Landegran et al., Science 241, 1077-80, 1988; Nakazawa et al., Proc. Natl. Acad. Sci. U.S.A. 91, 360-64, 1994), self-sustained sequence replication (Guatelli, et al., Proc. Natl. Acad. Sci. U.S.A. 87, 1874-78, 1990), transcriptional amplification system (Kwoh et al., Proc. Natl. Acad. Sci. U.S.A. 86, 1173-77, 1989), and Q-Beta Replicase (Lizardi et al., BioTechnology 6, 1197, 1988). Amplified molecules can be detected using techniques well known to those of skill in the art. These detection schemes are especially useful for the detection of nucleic acid molecules if such molecules are present in very low numbers.

Amplified nucleic acid molecules can then be assayed by any of a variety of methods to detect mutant SP-C alleles comprising a SNP. Assay methods include, but are not limited to: (1) allele-specific oligonucleotide probing, (2) differential restriction endonuclease digestion, (3) ligase-mediated gene detection (“LMGD”), (4) gel electrophoresis, (5) oligonucleotide ligation assay, (6) exonuclease-resistant nucleotides, and (7) genetic bit analysis. Other methods, such as fluorescence resonance energy transfer (“FRET”) (Wolf et. al., Proc. Natl. Acad. Sci. U.S.A. 85, 8790-94, 1988), also can be used.

Allele-Specific Oligonucleotide Probing (“ASO”)

Allele-specific oligonucleotide probing involves assaying nucleic acid of a subject for the presence or absence of a mutant SP-C allele by contacting the nucleic acid with an allele-specific oligonucleotide probe (“ASO probe”) under conditions suitable for the probe to hybridize with a mutant SP-C allele but not with a wild-type SP-C allele, and detecting the presence or absence of hybridization.

ASO probes can be complementary to either DNA or mRNA. The length of the probe is not critical. Under appropriate hybridization conditions, an ASO probe hybridizes only to a particular nucleic acid sequence that contains a SNP. The probes are detectably labeled so that hybridization with a SNP-containing sequence can be detected. Suitable detectable labels include fluorescent, chemiluminescent, and radioactive labels. If desired, probes can be modified to increase stability. See U.S. Pat. Nos. 5,176,996, 5,264,564, and 5,256,775).

Optionally, ASO probes can be attached to a solid support, such as a gene chip, glass or plastic slide, tissue culture plate, microtiter well, tube, silicon chip, or particle, such as a bead (e.g., a latex, polystyrene, or glass bead). Multiple probes can be attached to a solid support in an array, so that the identity of a particular SNP can be indicated by the position of the ASO probe in the array.

If desired, either amplified test nucleic acid or the ASO probe can be bound to each of two solid matrices (e.g., nylon or nitrocellulose membrane) and placed into separate hybridization reactions with an ASO probe or amplified nucleic acid, respectively. For example, if the amplified nucleic acid were bound onto a solid matrix, one hybridization reaction would utilize an oligonucleotide probe specific for a particular allele under conditions optimal for hybridization of this probe to its complement. The other hybridization reaction would utilize an oligonucleotide specific to another allele under conditions optimal for hybridization of that probe to its complement. Accordingly, the ASO probes may bear the same label, but will still be distinguishable because they are hybridized in separate chambers. This technique permits the determination of whether the subject's nucleic acid encodes the allele in question and also whether the subject is a heterozygote or a homozygote. If an ASO probe is found to bind to a subject's nucleic acid on only one membrane, then the subject is homozygous for that particular allele which the ASO probe was designed to bind. If the ASO probes are found to hybridize the subject's nucleic acid on both membranes, then the subject is heterozygous. An example of this technique applied to the detection of cystic fibrosis heterozygotes is described in Lemna et al., N.E.J.M. 322, 291-96, 1990.

Differential Restriction Endonuclease Digestion (“DRED”)

Another method of detecting a mutant SP-C allele uses a restriction endonuclease which cleaves a recognition site which is present in the wild-type SP-C allele but not in the mutant SP-C allele or vice versa. For example, the SNP c460+1G>A eliminates a recognition site for the enzyme BstNI. Thus, differential cleavage by this enzyme can distinguish SP-C alleles comprising that SNP from those that have a wild-type sequence at that position.

Gel Electrophoresis

Alterations in electrophoretic mobility can be used to identify a mutant SP-C allele. For example, single strand conformation polymorphism (SSCP) can be used to detect differences in electrophoretic mobility between mutant and wild type nucleic acids. See Orita et al., Proc. Natl. Acad. Sci. U.S.A. 86, 2766, 1989; Cotton, Mutation Res. 285, 125-44, 1993; and Hayashi, Genet. Anal. Tech. Appl. 973-79, 1992. Single-stranded DNA fragments of sample and control SP-C nucleic acids are denatured and allowed to renature. Because the secondary structure of single-stranded nucleic acids varies according to sequence, the resulting alteration in electrophoretic mobility enables the detection of even a single base change. The DNA fragments can be labeled or can be detected with labeled probes. The sensitivity of the assay can be enhanced by using RNA, the secondary structure of which is more sensitive than DNA to a change in sequence. Heteroduplex analysis can be used to separate double stranded heteroduplex molecules on the basis of changes in electrophoretic mobility. See Keen et al., Trends Genet. 7, 5, 1991.

Alternatively, movement of mutant or wild-type fragments in polyacrylamide gels containing a gradient of denaturant can be assayed (denaturing gradient gel electrophoresis, “DGGE”) (Myers et al., Nature 313, 495, 1985). In this method, DNA can be modified to ensure that it does not completely denature, for example by adding a GC clamp of approximately 40 bp of high-melting GC-rich DNA. Optionally, a temperature gradient can be used in place of a denaturing agent gradient to identify differences in the mobility of control and sample DNA (Rosenbaum & Reissner, Biophys. Chem. 265, 12753, 1987).

Oligonucleotide Ligation Assay (“OLA”)

Identification of a mutant SP-C allele also can be carried out using an oligonucleotide ligation assay (“OLA”) (see U.S. Pat. No. 4,998,617; Landegren et al., Science 241, 1077-80, 1988). The OLA protocol uses two oligonucleotides, which are designed to hybridize to abutting sequences of a single strand of a target. One of the oligonucleotides is linked to a separation marker (e.g., biotin), and the other comprises a detectable label. If the precise complementary sequence is found in a target molecule, the oligonucleotides will hybridize such that their termini abut and create a ligation substrate. Ligation then permits the labeled oligonucleotide to be recovered using avidin or another biotin ligand.

If desired, the target DNA can first be amplified. Nickerson et al., Proc. Natl. Acad. Sci. U.S.A. 87, 8923-27, 1990. Several techniques based on this OLA method have been developed and can be used to detect specific SP-C alleles. For example, U.S. Pat. No. 5,593,826 discloses use of an OLA using an oligonucleotide having a 3′-amino group and a 5′-phosphorylated oligonucleotide to form a conjugate having a phosphoramidate linkage. OLA combined with PCR permits typing of two alleles in a single microtiter well. Tobe et al., Nucleic Acids Res. 24, 3728, 1996. By marking each of the allele-specific primers with a unique hapten, i.e., digoxigenin and fluorescein, each OLA reaction can be detected by using hapten-specific antibodies that are labeled with different enzyme reporters, alkaline phosphatase, or horseradish peroxidase. This system permits the detection of the two alleles using a high throughput format that leads to the production of two different colors.

Ligase-Mediated Gene Detection (“LMGD”)

Ligase-mediated gene detection is taught in U.S. Pat. No. 6,008,335. Briefly, the method uses a pair of oligonucleotide probes that hybridize adjacent to each other, for example, at a specific nucleotide that distinguishes one SP-C allele from another SP-C allele. Each probe comprises a different detectable label. After hybridization to an allele-distinguishing segment, the two probes are ligated together. The ligated probes are then isolated from the segment and both labels are detected together, confirming the presence of an allele-specific nucleotide sequence.

Exonuclease-Resistant Nucleotides

A mutant SP-C allele also can be detected using a specialized exonuclease-resistant nucleotide (see U.S. Pat. No. 4,656,127). A primer complementary to the allelic sequence immediately 3′ to the polymorphic site to be detected is permitted to hybridize to a target molecule obtained from a test subject. If the polymorphic site on the target molecule contains a nucleotide that is complementary to the particular exonuclease-resistant nucleotide derivative present, that derivative will be incorporated onto the end of the hybridized primer. Such incorporation renders the primer resistant to exonuclease, permitting its detection. Because the identity of the exonuclease-resistant derivative of the sample is known, a primer that has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

A solution-based method can be used to determine the identity of a nucleotide of a polymorphic site. See French Patent 2,650,840 and WO 91/02087. In this method, a primer is used that is complementary to allelic sequences immediately 3′ to a polymorphic site. The method determines the identity of the nucleotide of that site using labeled dideoxynucleotide derivatives which, if complementary to the nucleotide of the polymorphic site, will become incorporated onto the terminus of the primer.

Genetic Bit Analysis (GBA™)

An alternative method, known as GBA™, is described in WO 92/15712. This method uses mixtures of labeled terminators and a primer that is complementary to the sequence 3′ to a polymorphic site. The labeled terminator that is incorporated is thus determined by, and complementary to, the nucleotide present in the polymorphic site of a target molecule. In contrast to the solution-based method described above, GBATM preferably is a heterogeneous phase assay, in which the primer or the target molecule is immobilized to a solid phase.

Protein Truncation Test

For polymorphisms that produce premature termination of protein translation, the protein truncation test offers an efficient diagnostic approach. See Roest et. al., Hum. Mol. Genet. 2, 1719-21, 1993; van der Luijt et. al., Genomics 20, 1-4, 1994. For this test, RNA is isolated from a test tissue and reverse-transcribed, then the segment of interest is amplified using PCR. The PCR products are then used as a template for nested PCR amplification with a primer that contains an RNA polymerase promoter and a sequence for initiating eukaryotic translation. After amplification of the region of interest, the unique motifs incorporated into the primer permit sequential in vitro transcription and translation of the PCR products. Upon sodium dodecyl sulfate-polyacrylamide gel electrophoresis of translation products, the appearance of truncated polypeptides signals the presence of a mutation that causes premature termination of translation. In a variation of this technique, DNA is used as the PCR template when the target region of interest is derived from a single exon.

Antibody Binding

Screening may also be based on the antigenic characteristics of a mutant SP-C protein, i.e., an SP-C protein comprising an amino acid alteration due to the presence of a SNP in the SP-C gene. Antibodies which specifically bind to such mutant proteins can be used to detect the presence of such proteins in immunoassays, such as Western blots, ELISAs, radioimmunoassays, immunohistochemical assays, immunoprecipitations, or other immunochemical assays known in the art.

All patents, patent applications, and references cited in this disclosure are expressly incorporated herein by reference. The above disclosure generally describes the present invention. A more complete understanding can be obtained by reference to the following specific examples, which are provided for purposes of illustration only and are not intended to limit the scope of the invention.

### EXAMPLE 1

Patients and Case Histories

Patient samples were received as part of a program to evaluate infants with lung disease of unknown etiology for mutations in the surfactant protein genes. The institutional review boards of the respective institutions approved protocols for these evaluations, and informed consent for genetic testing was obtained.

The case patient was a full-term Caucasian female whose mother had been diagnosed as having DIP at 1 year-of-age and had been treated with corticosteroids until age 15. The maternal grandfather had died from life-long lung disease of unknown etiology. Respiratory symptoms of tachypnea and cyanosis in room air developed at 6 weeks-of-age. Chest X-ray showed hyperinflation with increased interstitial markings. Because of the family history, open lung biopsy was performed. The histologic features were felt to most closely resemble cellular or non-specific interstitial pneumonitis (1, 11). The child was treated with supplemental oxygen and corticosteroids with some improvement in her respiratory symptoms. The mother's lung disease worsened postpartum, and she died from respiratory failure.

Control lung tissues were from unused donor lung tissue and from patients undergoing lung transplantation for end-stage pulmonary disease. These included a ventilator-dependent 2 year-old with bronchopulmonary dysplasia and adolescents with primary pulmonary hypertension12. Control DNA samples were from adult volunteers without a known history of lung disease (13).

### EXAMPLE 2

DNA Preparation and Analysis

Genomic DNA was prepared from blood leukocytes using a commercially available kit (Puregene, Gentra Systems, Minneapolis, Minn.). Amplimers spanning exons 1 to 2 (genomic positions −143 to 996) and exons 3 to 6 (g.1212-g.2522) of the SP-C gene were generated by PCR from genomic DNA and analyzed by direct sequencing of the PCR products. PCR conditions were the same as used for amplification of the SP-B gene, with an annealing temperature of 62° C., and cycle sequencing was performed as previously described (13). Patient SP-C gene sequences were compared to published SP-C sequences (14-16). Restriction analyses were performed on PCR products using reagents and conditions provided by the manufacturer (New England Biolabs, Beverly, Mass.). DNA from formalin-fixed paraffin embedded tissue was extracted using a microwave based method (17) and analyzed by PCR using a nested strategy, with 20 cycles first performed using primers spanning exon 4, and 2 ml of this reaction amplified by another 20 cycles in a reaction using primers internal to those in the first reaction.

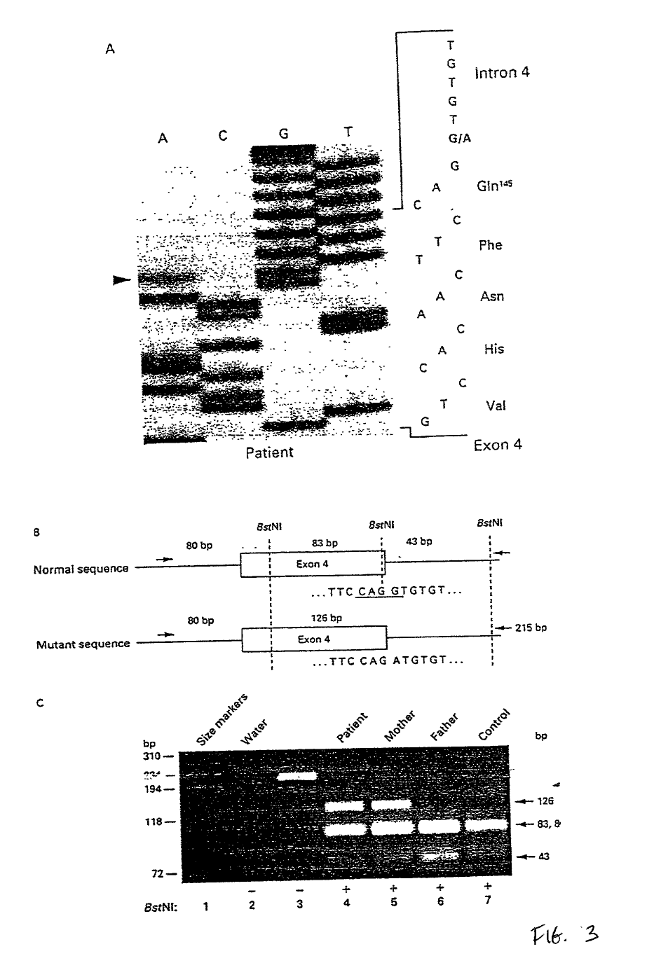

A heterozygous G to A transition was identified at the first base of intron 4 (g.1728, c.460+1G>A) of the case patient's SP-C gene, which would abolish the normal donor splice site (FIG. 3A). No other deviations from the published SP-C coding sequences or intron-exon boundaries were observed (14-16). This mutation eliminated a recognition site for the restriction enzyme BstN1. Restriction analysis confirmed the presence of the mutation in the case patient and her mother (FIG. 3B), but it was not found on 100 chromosomes from control subjects, indicating that it is not a common polymorphism.

Thus, we identified a mutation in the SP-C gene in two individuals from the same family who did not have respiratory symptoms at birth, but who subsequently developed interstitial lung disease. These observations suggest that while SP-C may not be critical for respiratory adaptation at birth, it is important for normal postnatal lung function, and that mutations in this gene may be associated with interstitial lung disease.

The SP-C mutation was identified on only one allele, consistent with the autosomal dominant pattern of inheritance, although occult mutations may have been present on the other alleles. The c.460+1G>A mutation resulted in production of an abnormal proprotein, and transcripts encoding normal proSP-C were present in amounts similar to those encoding the abnormal protein. These observations suggest that the abnormal protein had a dominant negative effect on SP-C function or metabolism and implicate abnormalities in SP-C metabolism and function in the pathogenesis of ILD.

### EXAMPLE 3

Protein Blotting, Immunohistochemistry, Electron Microscopy

SDS polyacrylamide gel electrophoresis and protein blotting were performed on homogenates of lung tissue frozen in liquid nitrogen at the time of biopsy and immunohistochemical analyses were performed on formalin-fixed, paraffin-embedded tissue as previously described (12, 13). When undetectable or low levels of staining were observed, antigen retrieval methods were used by immersing sections in sodium citrate buffer and microwaving them to enhance sensitivity or unmask epitopes hidden by fixation (18). Production and characterization of polyclonal antisera directed against SP-A, SP-B, proSP-B, and proSP-C has been described (19-22). Anti-mature SP-C (Byk-Gulden, Konstanz, Germany) was generated against recombinant human SP-C and used as described (23). A commercial monoclonal antibody, CD68, was used for the detection of human macrophages (Dako Corporation, Carpinteria, Calif.). Small pieces of snap frozen lung tissue were thawed quickly in fixative at room temperature and prepared for electron microscopy as previously described (24).

Histopathology findings for the case patient included well preserved pulmonary architecture, alveolar type II cell hyperplasia, and an interstitial infiltrate comprised mostly of mature lymphocytes with scattered myofibroblasts. Some non-inflated alveoli were filled with desquamated cells, the majority of which were immunopositive for the macrophage cell marker CD68. Normal appearing lamellar bodies were observed in alveolar type II cells by electron microscopy. The patient's mother had areas of diffuse fibrosis and honeycombing, with areas of mild interstitial lymphocytic infiltration that were patchy in distribution, accumulations of alveolar macrophages, and areas of superimposed alveolar damage.

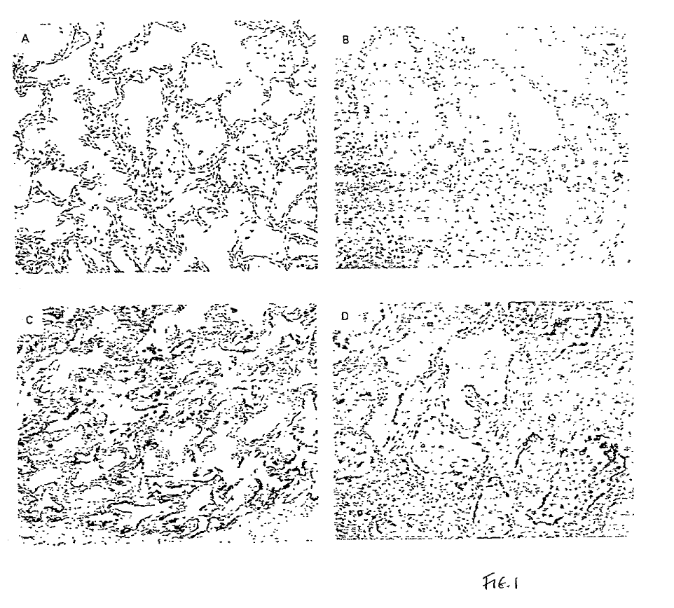

Immunostaining for proSP-C was absent in biopsy lung tissue of the case patient, and was extremely weak or completely absent in most regions of autopsy lung tissue from her mother (FIG. 1). After antigen retrieval immunostaining for proSP-C was readily detected, indicating that the proteins were present, although possibly reduced in amount. ProSP-C staining was restricted to alveolar Type II cells and not detected in luminal material. SP-A, mature SP-B, and proSP-B staining was observed in alveolar Type II cells in each patient, along the alveolar surface, in association with alveolar macrophages and in intra-alveolar exudates.

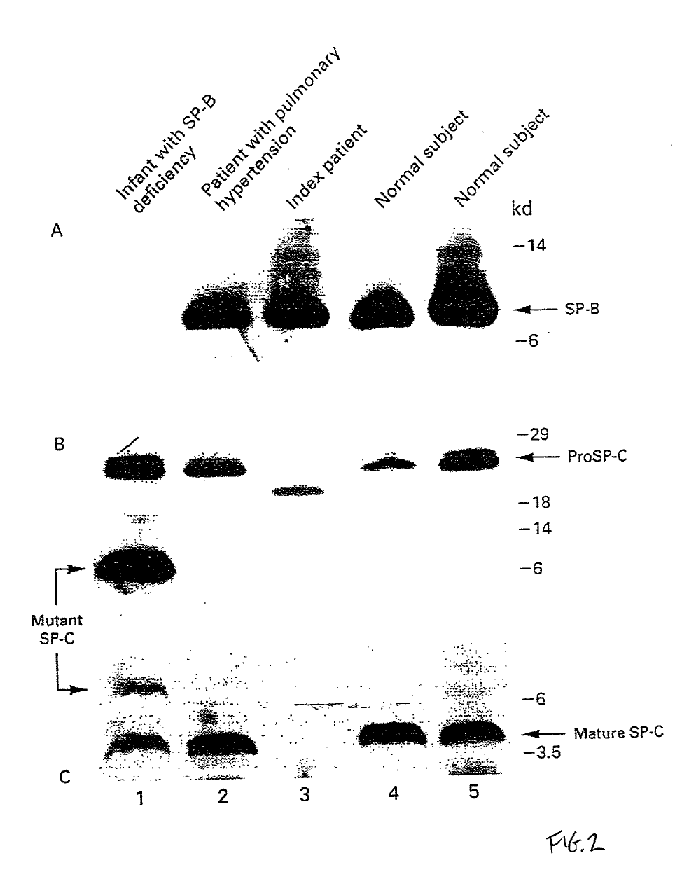

ProSP-C was reduced in amount in the case patient's lung tissue, and the predominant proSP-C band migrated at a lesser molecular weight compared to controls (FIG. 2). Mature SP-C was undetectable in lung tissue and in bronchoalveolar lavage fluid (BALF) from the case patient, but was readily detected in BALF obtained from age matched control patients. Mature SP-B was present in amounts comparable to controls as were SP-A and proSP-B.

The lack of mature SP-C in lung tissue and BALF from the case patient supports the hypothesis that proSP-C was not properly processed and secreted.

### EXAMPLE 4

RNA Analysis

RNA was prepared from frozen lung tissue as previously described (13), and five mg reverse transcribed using Superscript II (BRL Life Technologies, Gaithersburg, Md.) with an oligo dT primer using reagents and conditions supplied by the manufacturer. SP-C cDNA was generated using primers corresponding to cDNA nucleotides 15 to 32 (forward) and 715 to 698 (reverse). PCR conditions were the same as used for amplification of SP-B cDNA (13).

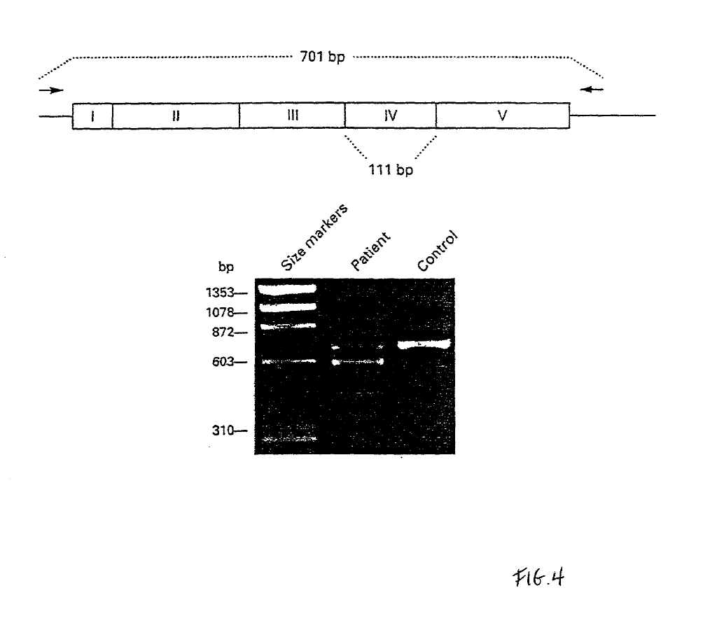

SP-C RT-PCR products of the expected size and one shorter by approximately 110 bp were amplified from RNA prepared from the case patient's lung tissue (FIG. 4). Sequence analysis indicated that the shorter RT-PCR product lacked the sequence corresponding to exon 4. Analysis of single nucleotide polymorphisms in the SP-C gene indicated that the shorter transcripts were derived from the allele with the c.460+1G>A substitution. No other deviations from the published SP-C sequences were observed (14, 16).

### REFERENCES

1. Katzenstein A L, Myers J L. Nonspecific interstitial pneumonia and the other idiopathic interstitial pneumonias: classification and diagnostic criteria [editorial]. Am J Surg Pathol 2000; 24:1-3.

2. Howenstine M S, Eigen H. Current concepts on interstitial lung disease in children. Curr Opin Pediatr 1999; 11:200-4.

3. Fan L L, Langston C. Chronic interstitial lung disease in children. Pediatr Pulmonol 1993; 16:184-96.

4. Fan L L, Mullen A L, Brugman S M, Inscore S C, Parks D P, White C W. Clinical spectrum of chronic interstitial lung disease in children. J Pediatr 1992; 121:867-72.

5. Nicholson A G, Kim H, Corrin B, et al. The value of classifying interstitial pneumonitis in childhood according to defined histological patterns. Histopathology 1998; 33:203-11.

6. Buchino J J, Keenan W J, Algren J T, Bove K E. Familial desquamative interstitial pneumonitis occurring in infants. Am J Med Genet Suppl 1987; 3:285-91.

7. Stillwell P C, Norris D G, O'Connell E J, Rosenow E Cd, Weiland L H, Harrison E G, Jr. Desquamative interstitial pneumonitis in children. Chest 1980; 77:165-71.

8. Katzenstein A L, Gordon L P, Oliphant M, Swender P T. Chronic pneumonitis of infancy. A unique form of interstitial lung disease occurring in early childhood. Am J Surg Pathol 1995; 19:439-47.

9. Kavantzas N, Theocharis S, Agapitos E, Davaris P. Chronic pneumonitis of infancy. An autopsy study of 12 cases. Clin Exp Pathol 1999; 47:96-100.

10. Sharief N, Crawford O F, Dinwiddie R. Fibrosing alveolitis and desquamative interstitial pneumonitis. Pediatr Pulmonol 1994; 17:359-65.

11. Schroeder S A, Shannon D C, Mark E J. Cellular interstitial pneumonitis in infants. A clinicopathologic study. Chest 1992; 101:1065-9.

12. Nogee L M, de Mello D E, Dehner L P, Colten H R. Brief report: deficiency of pulmonary surfactant protein B in congenital alveolar proteinosis. N Engl J Med 1993; 328:406-10.

13. Nogee L M, Wert S E, Proffit S A, Hull W M, Whitsett J A. Allelic Heterogeneity in Hereditary Surfactant Protein B (SP-B) Deficiency. Am J Respir Crit Care Med 2000; 161:973-981.

14. Glasser S W, Korfhagen T R, Weaver T E, et al. cDNA, deduced polypeptide structure and chromosomal assignment of human pulmonary surfactant proteolipid, SPL(pVal). J Biol Chem 1988; 263:9-12.

15. Glasser S W, Korfhagen T R, Perme C M, Pilot-Matias T J, Kister S E, Whitsett J A. Two SP-C genes encoding human pulmonary surfactant proteolipid. J Biol Chem 1988; 263:10326-31.

16. Warr R G, Hawgood S, Buckley D I, et al. Low molecular weight human pulmonary surfactant protein (SP5): isolation, characterization, and cDNA and amino acid sequences. Proc Natl Acad Sci U S A 1987; 84:7915-9.

17. Banerjee S K, Makdisi W F, Weston A P, Mitchell S M, Campbell D R. Microwave-based DNA extraction from paraffin-embedded tissue for PCR amplification. Biotechniques 1995; 18:768-70, 772-3.

18. Leong AS-Y, Milios J. An assessment of the efficacy of the microwave antigen retrieval procedure on a range of tissue antigens. Appl Immunohistochem 1993; 14:267-274.

19. Stahlman M T, Gray M E, Ross G F, et al. Human surfactant protein-A contains blood group A antigenic determinants. Pediatr Res 1992; 31:364-71.

20. Baatz J E, Elledge B, Whitsett J A. Surfactant protein SP-B induces ordering at the surface of model membrane bilayers. Biochemistry 1990; 29:6714-20.

21. Lin S, Akinbi H T, Breslin J S, Weaver T E. Structural requirements for targeting of surfactant protein B (SP-B) to secretory granules in vitro and in vivo. J Biol Chem 1996; 271:19689-95.

22. Vorbroker D K, Profitt S A, Nogee L M, Whitsett J A. Aberrant processing of surfactant protein C in hereditary SP-B deficiency. Am J Physiol 1995; 268:L647-56.

23. Ross G F, Ikegami M, Steinhilber W, Jobe A H. Surfactant protein C in fetal and ventilated preterm rabbit lungs. Am J Physiol 1999; 277:L1104-8.

24. Na C-L, Beck D C, Breslin J S, Wert S E, Weaver T E. An en bloc staining protocol improves the preservation of lamellar bodies in alveolar type II cells for transgenic mice expressing modified pulmonary surfactant protein B. Microsc Microanal 1998; 4:852-853.

25. Farrell P M, Avery M E. Hyaline membrane disease. Am Rev Respir Dis 1975; 111:657-88.

26. Whitsett J A, Ohning B L, Ross G, et al. Hydrophobic surfactant-associated protein in whole lung surfactant and its importance for biophysical activity in lung surfactant extracts used for replacement therapy. Pediatr Res 1986; 20:460-7.

27. Clark J C, Wert S E, Bachurski C J, et al. Targeted disruption of the surfactant protein B gene disrupts surfactant homeostasis, causing respiratory failure in newborn mice. Proc Natl Acad Sci U S A 1995; 92:7794-8.

28. Keller A, Eistetter H R, Voss T, Schafer K P. The pulmonary surfactant protein C (SP-C) precursor is a type II transmembrane protein. Biochem J 1991; 277:493-9.

29. Keller A, Steinhilber W, Schafer K P, Voss T. The C-terminal domain of the pulmonary surfactant protein C precursor contains signals for intracellular targeting. Am J Respir Cell Mol Biol 1992; 6:601-8.

30. Beers M F, Lomax C A, Russo S J. Synthetic processing of surfactant protein C by alevolar epithelial cells. The COOH terminus of proSP-C is required for post-translational targeting and proteolysis. J Biol Chem 1998; 273:15287-93.

31. Baatz J E, Smyth K L, Whitsett J A, Baxter C, Absolom D R. Structure and functions of a dimeric form of surfactant protein SP-C: a Fourier transform infrared and surfactometry study. Chem Phys Lipids 1992; 63:91-104.

32. Johnston J A, Ward C L, Kopito R R. Aggresomes: a cellular response to misfolded proteins. J Cell Biol 1998; 143:1883-98.

33. Whitseff J A, Weaver T E, Clark J C, et al. Glucocorticoid enhances surfactant proteolipid Phe and pVal synthesis and RNA in fetal lung. J Biol Chem 1987; 262:15618-23.

34. Bross P, Corydon T J, Andresen B S, Jorgensen M M, Bolund L, Gregersen N. Protein misfolding and degradation in genetic diseases. Hum Mutat 1999; 14:186-98.

35. Rubenstein R C, Egan M E, Zeitlin P L. In vitro pharmacologic restoration of CFTR-mediated chloride transport with sodium 4-phenylbutyrate in cystic fibrosis epithelial cells containing delta F508-CFTR. J Clin Invest 1997; 100:2457-65.

36. Burrows J A, Willis L K, Perlmutter D H. Chemical chaperones mediate increased secretion of mutant alpha 1-antitrypsin (alpha 1-AT) Z: A potential pharmacological strategy for prevention of liver injury and emphysema in alpha 1-AT deficiency. Proc Natl Acad Sci U S A 2000; 97:1796-801.

1. A purified preparation of antibodies which specifically bind to a mutant human surfactant protein C comprising an amino acid alteration due to the presence of a single nucleotide polymorphism (SNP) in a gene encoding the mutant surfactant protein C, wherein the SNP is associated with interstitial lung disease, wherein the antibodies do not bind to a wild-type human surfactant protein C.

2. The preparation of claim 1 wherein the SNP is located at a nucleotide position of SEQ ID NO:1 selected from the group consisting of nucleotide positions 114, 219, 243, 246, 324, 332, 335, 359, 369, 402, 443-445, the intronic nucleotide immediately 3′ of nucleotide 460 (460+1), 521-523, 585, and 588.

3. The preparation of claim 1 wherein the SNP is located at nucleotide position 460+1 of SEQ ID NO:1.

4. The preparation of claim 3 wherein the nucleotide at position 460+1 is adenine.

5. The preparation of claim 3 wherein the nucleotide at position 460+1 is thymidine.

6. The preparation of claim 1 wherein the antibodies are polyclonal antibodies.

7. The preparation of claim 1 wherein the antibodies are monoclonal antibodies.

8. The preparation of claim 1 wherein the antibodies are Fab, F(ab′)₂, or Fv fragments.

9. A single-stranded polynucleotide comprising 12 contiguous nucleotides of a mutant allele of a human surfactant protein C gene, wherein the 12 contiguous nucleotides comprise a SNP associated with interstitial lung disease.

10. The single-stranded polynucleotide of claim 9 wherein the SNP is located at a nucleotide position of SEQ ID NO:1 selected from the group consisting of nucleotide positions 49, 114, 219, 243, 246, 324, 332, 335, 359, 369, 402, 443-445, the intronic nucleotide immediately 3′ of nucleotide 460 (460+1), 521-523, 585, and 588.

11. The single-stranded polynucleotide of claim 9 wherein the SNP is located at nucleotide position 460+1 of SEQ ID NO:1.

12. The single-stranded polynucleotide of claim 11 wherein the nucleotide at position 460+1 is adenine.

13. The single-stranded polynucleotide of claim 11 wherein the nucleotide at position 460+1 is thymidine.

14. The single-stranded polynucleotide of claim 9 which comprises a detectable label.

15. The single-stranded polynucleotide of claim 9 wherein the SNP is at either the 3′ or the 5′ end of the polynucleotide.

16. The single-stranded polynucleotide of claim 9 which is bound to a solid support.

17. A kit, comprising:
a reagent for detecting a SNP in a mutant allele of a human surfactant protein C gene, wherein the SNP is associated with interstitial lung disease; and
instructions for a method of detecting the SNP.

18. The kit of claim 17 wherein the reagent is an antibody which specifically binds to a mutant human surfactant protein C comprising an amino acid alteration due to the presence of the SNP, wherein the antibody does not bind to a wild-type human surfactant protein C.

19. The kit of claim 17 wherein the reagent is a single-stranded polynucleotide comprising 12 contiguous nucleotides of the mutant allele, wherein the 12 contiguous nucleotides comprise the SNP or the complement of the SNP.

20. The kit of claim 17 wherein the SNP is located at a nucleotide position of SEQ ID NO:1 selected from the group consisting of nucleotide positions 49, 114, 219, 243, 246, 324, 332, 335, 359, 369, 402, 443-445, the intronic nucleotide immediately 3′ of nucleotide 460 (460+1), 521-523, 585, and 588.

21. The kit of claim 17 wherein the SNP is located at nucleotide position 460 +1 of SEQ ID NO:1.

22. The kit of claim 21 wherein the nucleotide at position 460+1 is adenine.

23. The kit of claim 21 wherein the nucleotide at position 460+1 is thymidine.

24. A method of identifying an individual as predisposed to developing interstitial lung disease associated with a defect in surfactant protein C, comprising the steps of:
assaying a biological sample obtained from the individual to determine if an allele of a surfactant protein C gene comprises a SNP associated with interstitial lung disease; and
identifying the individual as predisposed to developing the interstitial lung disease if the allele comprises the SNP.

25. The method of claim 24 wherein the biological sample is lung tissue.

26. The method of claim 24 wherein the biological sample is bronchoalveolar lavage fluid.

27. The method of claim 24 wherein the biological sample is blood.

28. The method of claim 24 wherein the SNP is located at a nucleotide position of SEQ ID NO:1 selected from the group consisting of nucleotide positions 49, 114, 219, 243, 246, 324, 332, 335, 359, 369, 402, 443-445, the intronic nucleotide immediately 3′ of nucleotide 460 (460+1), 521-523, 585, and 588.

29. The method of claim 24 wherein the SNP is located at nucleotide position 460+1 of SEQ ID NO:1.

30. The method of claim 29 wherein the nucleotide at position 460+1 is adenine.

31. The method of claim 29 wherein the nucleotide at position 460+1 is thymidine.

32. The method of claim 24 wherein surfactant protein C in the biological sample is assayed to detect an amino acid alteration due to the presence of the SNP.

33. The method of claim 32wherein the surfactant protein C is assayed using an antibody which specifically binds to a mutant surfactant protein C comprising the amino acid alteration, wherein the antibody does not bind to a wild-type surfactant protein C.

34. The method of claim 25 wherein nucleic acid is assayed to detect the SNP.

35. The method of claim 34 wherein the nucleic acid is assayed using a single-stranded polynucleotide comprising 12 contiguous nucleotides of a mutant allele of the surfactant protein C gene, wherein the 12 contiguous nucleotides comprise the SNP or the complement of the SNP.

36. The method of claim 25 wherein the interstitial lung disease is desquamative interstitial pneumonitis.

37. A method of diagnosing interstitial lung disease associated with a defect in surfactant protein C, comprising the steps of:
assaying a biological sample obtained from an individual to determine if an allele of a surfactant protein C gene comprises a SNP associated with interstitial lung disease; and
identifying the individual as having the interstitial lung disease if the allele comprises the SNP.

38. The method of claim 37 wherein the biological sample is lung tissue.

39. The method of claim 37 wherein the biological sample is bronchoalveolar lavage fluid.

40. The method of claim 37 wherein the biological sample is blood.

41. The method of claim 37 wherein the SNP is located at a nucleotide position of SEQ ID NO:1 selected from the group consisting of nucleotide positions 49, 114, 219, 243, 246, 324, 332, 335, 359, 369, 402, 443-445, the intronic nucleotide immediately 3′ of nucleotide 460 (460+1), 521-523, 585, and 588.

42. The method of claim 37 wherein the SNP is located at nucleotide position 460+1 of SEQ ID NO:1.

43. The method of claim 42 wherein the nucleotide at position 460+1 is adenine.

44. The method of claim 42 wherein the nucleotide at position 460+1 is thymidine.

45. The method of claim 37 wherein surfactant protein C in the biological sample is assayed to detect an amino acid alteration due to the presence of the SNP.

46. The method of claim 45 wherein the surfactant protein C is assayed using an antibody which specifically binds to a mutant surfactant protein C comprising the amino acid alteration, wherein the antibody does not bind to a wild-type surfactant protein C.

47. The method of claim 37 wherein nucleic acid is assayed to detect the SNP.

48. The method of claim 47 wherein the nucleic acid is assayed using a single-stranded polynucleotide comprising 12 contiguous nucleotides of a mutant allele of the surfactant protein C gene, wherein the 12 contiguous nucleotides comprise the SNP or the complement of the SNP.

49. A method of determining whether an individual having interstitial lung disease is likely to respond to a therapeutic intervention, comprising the steps of:
assaying a biological sample obtained from the individual to determine whether both alleles of the individual's surfactant protein C gene comprise a SNP associated with interstitial lung disease; and
identifying the individual as likely to respond to the therapeutic intervention if neither allele comprises the SNP.

50. The method of claim 49 wherein the biological sample is lung tissue.

51. The method of claim 49 wherein the biological sample is bronchoalveolar lavage fluid.

52. The method of claim 49 wherein the biological sample is blood.

53. The method of claim 49 wherein the SNP is located at a nucleotide position of SEQ ID NO:1 selected from the group consisting of nucleotide positions 49, 114, 219, 243, 246, 324, 332, 335, 359, 369, 402, 443-445, the intronic nucleotide immediately 3′ of nucleotide 460 (460+1), 521-523, 585, and 588.

54. The method of claim 49 wherein the SNP is located at nucleotide position 460+1 of SEQ ID NO:1.

55. The method of claim 54 wherein the nucleotide at position 460+1 is adenine.

56. The method of claim 54 wherein the nucleotide at position 460+1 is thymidine.

57. The method of claim 49 wherein the therapeutic intervention is administration of a glucocorticoid.

58. The method of claim 49 wherein the therapeutic intervention is administration of chloroquine.
